(19) United States
(12) Patent Application Publication
(10) Pub. No.: US 2009/0182371 A1
(43) Pub. Date: Jul. 16, 2009

(54) Implantable vascular filters, apparatus and methods of implantation

(75) Inventors: Jacob Lund Clausen, Lyngby (DK); Per Hendriksen, Herlufmagle (DK); Bruce R. Fleck, Indianapolis, IN (US); Dan Jones, Valrico, FL (US); Blayne A. Roeder, Lafayette, IN (US)

Correspondence Address: COOK GROUP PATENT OFFICE, P.O. BOX 2269, BLOOMINGTON, IN, 47402, US

(73) Assignees: William Cook Europe ApS, Bjaeverskov (DK); Cook Incorporated, Bloomington, IN (US); MED Institute, Inc., West Lafayette, IN (US)

(21) Appl. No.: 12/220,758

(22) Filed: Jul. 28, 2008

Related U.S. Application Data
(60) Provisional application No. 60/962,071, filed on Jul. 26, 2007.

(51) Int. Cl. A61F 2/01

(52) U.S. Cl. 606/200

(57) ABSTRACT

An implantable vascular filter and method of implantation where the filter is deployed at or near an implantation site and is then radially expanded from its radially compressed configuration under the action of a force controlled remotely by a surgeon, such as by expansion of a balloon in contact with the filter or by applying a force on the filter with the introducer; the expansion is controlled using a means such as control wire, or fluid injector so that the filter adopts a filtering configuration and securely engages with a vessel wall.

### CROSS-REFERENCE TO RELATED APPLICATIONS

This application claims priority of provisional application Ser. No. 60/962,071, filed Jul. 26, 2007.

### TECHNICAL FIELD

This disclosure generally relates to medical devices and in implantable vascular filters and to apparatus and methods of implantation therefore.

### BACKGROUND OF THE INVENTION

Filtering devices that are percutaneously implanted in the vena cava have been available for over 30 years. Percutaneous techniques are characterised by gaining access to an organ—in this case the vena cava—via a needle puncture of the skin rather than open surgery. This is particularly beneficial as the need for vena cava filtering devices arises in trauma patients, orthopaedic surgery patients, neuro-surgery patients, and immobile patients such as those requiring bed-rest or non-movement. As percutaneous implantation is far less invasive than open surgery it represents a substantially decreased risk of complications to the patient and also results in generally decreased recovery times after surgery. Percutaneous methods do, however, require low-profile prostheses.

A need for filtering devices arises due to a risk of embolism where an object within one part of the vasculature migrates to and causes a blockage in another part. Within these classes of patients there is significant risk of blood clots or thrombi forming within the peripheral vasculature. Such thrombi may detach and be carried by the circulation system to the lungs, thus causing a pulmonary embolism. As all blood flow from the peripheral vasculature returns to the heart via the vena cava, filters are implanted therein to substantially prevent the migration of emboli.

Implantable filtering devices may be designed to remain within the vasculature for the life of the patient. They may be adapted to be retrieved in a further procedure or constructed so as to be biodegradable. Implantable devices are distinct from temporary filters which are introduced into and remain within the vasculature only during a surgical procedure. For example, temporary filtering may occur whilst removing plaque from the interior of a blood vessel so as to prevent so-dislodged plaque from causing an embolism. Thus a temporary filter may be deployed downstream of the angioplasty to remove emboli from the blood stream. Such temporary filtering devices remain, broadly speaking, under the control of the surgeon while within the patient.

Current implantable filters are typically self-expandable. Suitable materials for self expandable filters include nitinol (trade name for a Nickel Titanium alloy), stainless steel and conichrome™—a cobalt-chromium-nickel-molybdenum-iron-alloy. Nitinol belongs to a group of materials known as shape memory alloys. Self expandable filters are introduced over a guide wire in a compressed state. As the filter exits the introducer it expands and contacts the wall of the body lumen. This may in one example be a resilient expansion of a device physically held in a compressed form within the introducer, and in another example a temperature controlled shape memory effect. Once the filter has left the introducer the expansion is uncontrolled by the surgeon and the filter will inevitably and immediately move into engagement with the vessel walls. Repositioning, where it is even possible, will then require disengagement from the vessel walls, which may damage the endothelial cells. Moreover, many permanent filters may only be introduced by one end and removed by the other, making such repositioning at the very least extremely undesirable as it will require a further access route to the vessel from the opposite end.

Further, with self-expandable filters there is no control exercisable by the surgeon over the expanded size of the filter. Furthermore the radially outward force they apply to the walls of a particular vessel depends on factors determined during manufacture such as the size of the expanded configuration, and the material and shape of the filter. Filters with insufficient outward radial force for a particular vessel may detach from the walls, whereas filters providing excessive force may over-stress the vessel walls and risk damage to or even puncture of the vessel wall. In many cases it is difficult or impracticable to provide filters that are matched with sufficient accuracy to a particular vasculature.

Therefore there exists a need for a filter which may be repositioned within the vasculature after it has left the introducer. There also exists a need for a filter whose deployed configuration may be matched by a surgeon to the diameter or other characteristics of a vessel.

### SUMMARY OF THE INVENTION

Disclosed herein is a filter for implantation within the vasculature, the filter comprising a structure adapted under the action of a force applied remotely of the filter to move after deployment from a radially compressed delivery configuration to a radially expanded filtering configuration in which it is in locating engagement with a vessel wall. Preferably, the structure undergoes plastic deformation in moving from the delivery configuration to the filtering configuration. Such a filter will also allow a much greater range of materials to be used in its construction, as elastic properties are unnecessary. It is envisaged that materials such as magnesium alloys, stainless steel and bulk metallic glasses may be used in its manufacture.

Preferably, the structure is adapted such that the radius of the filter is controllable in the application of said force. Thus, the surgeon may adapt the size of the filter to match the properties of a particular vessel, so avoiding over-stressing the vessel walls.

There is also disclosed a method of implantation of a filter within the vasculature, the filter comprising a structure adapted under the action of a force applied remotely of the filter to move after deployment from a radially compressed delivery configuration to a radially expanded filtering configuration in which it is in locating engagement with a vessel wall, the method comprising the steps of deploying the filter in the delivery configuration to or near an implantation site in the vasculature and by intervention of an attendant applying a force to move the structure from the delivery configuration to the filtering configuration.

Further objects, features and advantages of this invention will become readily apparent to persons skilled in the art after a review of the following description, with reference to the drawings and claims that are appended to and form a part of this specification.

The inventive filter is inserted into body lumens by way of an introducer. Where reference is made to the distal direction, it should be taken to mean the direction along the length of the body lumens in question away from the introducer and surgeon. Accordingly, where reference is made to the proximal direction, it should be taken to mean the direction along the length of the body lumens towards the introducer and surgeon. Where reference is made to radial or circumferential directions, these are defined with respect to the longitudinal axis of the body lumens.

### BRIEF DESCRIPTION OF THE DRAWING

FIGS. 1a, 1b and 1c show a filter in accordance with a first embodiment of the present invention in stages of increasing expansion.

FIG. 2 shows a filter in accordance with a further embodiment of the present invention.

FIGS. 3a, 3b and 3c show selected struts of a filter in stages of increasing expansion in accordance with the embodiment of the present invention shown in FIGS. 1a to 1c.

FIG. 4 shows a method for attaching the distal ends of struts of a filter in accordance with the embodiment of the present invention shown in FIGS. 1a to 1c.

FIG. 5 shows a further method for attaching the distal ends of struts of a filter in accordance with the embodiment of the present invention shown in FIGS. 1a to 1c.

FIG. 6 shows a detailed view of an optional safety feature incorporated in a filter in accordance with the embodiment of the present invention shown in FIGS. 1a to 1c.

FIG. 7 shows a filter in accordance with a still further embodiment of the present invention in a collapsed state.

FIG. 8 shows the filter of FIG. 7 in an expanded state.

FIG. 9 shows a filter of FIGS. 7 and 8 deployed within body lumens.

FIG. 10 shows a filter in accordance with yet a further embodiment of the present invention in a collapsed state within an introducer.

FIG. 11 shows the filter of FIG. 10 in a semi-expanded state having been deployed from an introducer.

FIG. 12 shows a method of expansion for the filter of FIG. 10. FIG. 13 shows an optional modification to an introducer to aid detachment of the filter of FIG. 10.

### DETAILED DESCRIPTION

There will be described below in more detail apparatus for the implantation of a filter within the vasculature, comprising: an implantable filter having a structure, said structure being adapted such that under the action of a force applied remotely of the filter it will move after deployment within the vasculature from a radially compressed delivery configuration facilitating navigation through the vasculature, to a radially expanded filtering configuration in which it is in locating engagement with a vessel wall; and means for applying said force remotely of the filter to move said structure from said delivery configuration to said filtering configuration. The structure may undergo plastic deformation in moving from the delivery configuration to the filtering configuration. For example, a strut may be deformed beyond an elastic limit. The structure may be adapted such that the radius of the filter in said filtering configuration is controlled by the application of said force.

The means for applying force remotely of the filter may comprise an inflatable element temporarily located within said structure and means for the injection of fluid thereinto or a control member moveable in compression or in tension.

The radially compressed delivery configuration of the filter may be relaxed and temperature invariant. The filter is not held in a resiliently compressed form within a delivery sheath and reliance is not placed on temperature related shape memory effects. The structure may be adapted to move over-centre between the delivery configuration and the filtering configuration.

There will further be described a method of implantation of a filter within the vasculature, the filter comprising a structure adapted under the action of a force applied remotely of the filter to move after deployment from a radially compressed delivery configuration to a radially expanded filtering configuration in which it is in locating engagement with a vessel wall, the method comprising the steps of deploying the filter in the delivery configuration to or near an implantation site in the vasculature and by intervention of an attendant applying a force to move the structure from the delivery configuration to the filtering configuration. The structure may be plastically deformed in moving from the delivery configuration to the filtering configuration. Controlling the applied force may control the radius of the filter.

An introducer comprising a sheath and a control element may be introduced at least in part into a patient's vasculature, said sheath having a lumen and a distal and a proximal end, and harbouring at said distal end a filter in a radially compressed configuration within said lumen. The distal end of the sheath may be advanced through the vasculature of said patient to a position proximal a deployment location. The filter may be advanced relative to said sheath so that filter moves beyond the distal end of said sheath and is deployed from the lumen of said sheath. A portion of said control element may be actuated exterior to said patient so as to cause said filter to adopt said radially expanded configuration.

An embolic protection filter may comprise a plurality of struts, each having a proximal and a distal end and being attached at their respective distal ends at a filter hub; a support member having a first and a second end, said first end being pivotally attached to a point disposed along the length of a first of said plurality of struts and said second end being slidably attached to a second of said plurality of struts; a flexible pulling member, having a distal and a proximal end, and being attached at its distal end to said support strut at a point spaced away from said first end, so that the application of a force in tension to the pulling member at the proximal end causes the proximal ends of the respective support struts to move apart from one another. Application of a force in tension to the pulling member may causes the second end of the support member to slide along the second of the struts to an over centre location along said second of the struts.

Apparatus for the implantation of a filter within the vasculature may comprise an embolic protection filter and an introducer, wherein said filter comprises a plurality of struts, each joined at one end in a filter hub, said struts together serving in use to capture emboli; said filter having a radially compressed delivery configuration and a radially expanded filtering configuration and being so adapted that a force external of the filter is required to move the filter from the radially compressed delivery configuration to the radially expanded filtering configuration; wherein said introducer has a distal and a proximal end and comprises: a sheath having a lumen between a proximal and a distal end; and a flexible control member mounted within said sheath, having a distal and a proximal end, wherein said control member is releasably attached at its distal end operable to said filter hub; the filter being harboured within the distal end of the sheath such that relative longitudinal movement in a first sense between the proximal end of the sheath and the proximal end of the control member serves to deploy the filter from the movement and relative longitudinal movement in a second opposite sense between the proximal end of the sheath and the proximal end of the control member serves to move the filter to the radially expanded filtering configuration.

In one described method for implanting an embolic protection filter at a deployment location within a patient's vasculature, the filter has an annular vessel engaging portion, having a delivery configuration in which the diameter of said portion is reduced. The method comprises the steps of introducing at least a portion of an introducer into said patient's vasculature, said introducer comprising a sheath and an inflatable member, said sheath having a lumen and a distal and a proximal end, said lumen at said distal end containing said embolic protection filter; advancing said distal end of said sheath through the vascu1ture of said patient to a position proximal to the deployment location; advancing said filter distally re1ative to said sheath so that said filter moves beyond the distal end of said sheath and is deployed from the lumen of said sheath and inflating said inflatable member so as to increase the diameter of said vessel engaging portion of filter by an amount controlled by the degree of inflation of the inflatable member to bring said vessel engaging portion of filter into securing engagement with a vessel wall.

FIGS. 1 and 3 to 6 show a first embodiment of the present invention where a thin wire or suture (101) is attached to the filter. By pulling on these wires the filter is controllably expanded from a collapsed configuration as shown in FIG. 1a to an expanded configuration as shown in FIG. 1c. The filter (100) comprises a plurality of main struts (102) attached to a hub (103) at their respective distal ends, and a corresponding plurality of support struts (104), each support strut being hinged at one end with the freely moving end of a main strut and slidably attached at the other end to a neighbouring main strut. During expansion, the suture pulls the sliding end of the support strut proximally so as to force the two main struts apart. The suture extends along the length of the sheath used to introduce the filter (not shown) so that the expansion of the balloon may be controlled remotely of the delivery location by the surgeon.

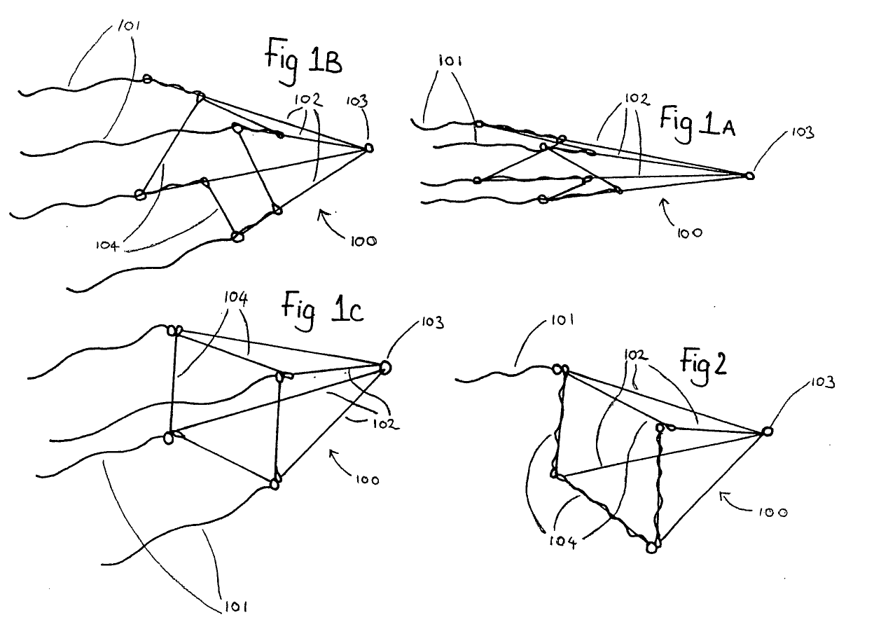

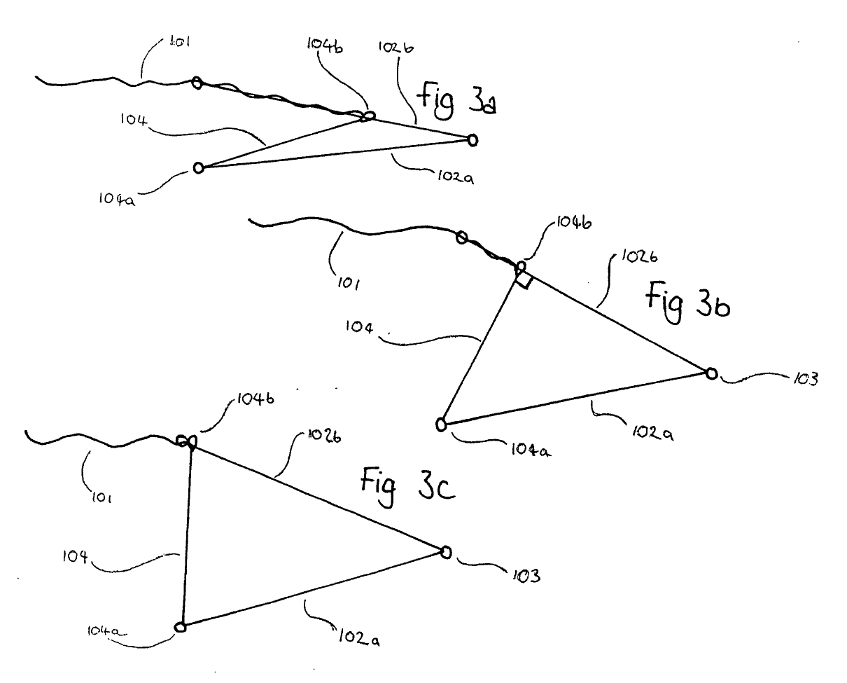

FIG. 2 shows further embodiment, substantially similar to the filter of FIG. 1, where the ends of all support struts are linked by a single suture (101) so that the pulling force need only be applied to a single suture.

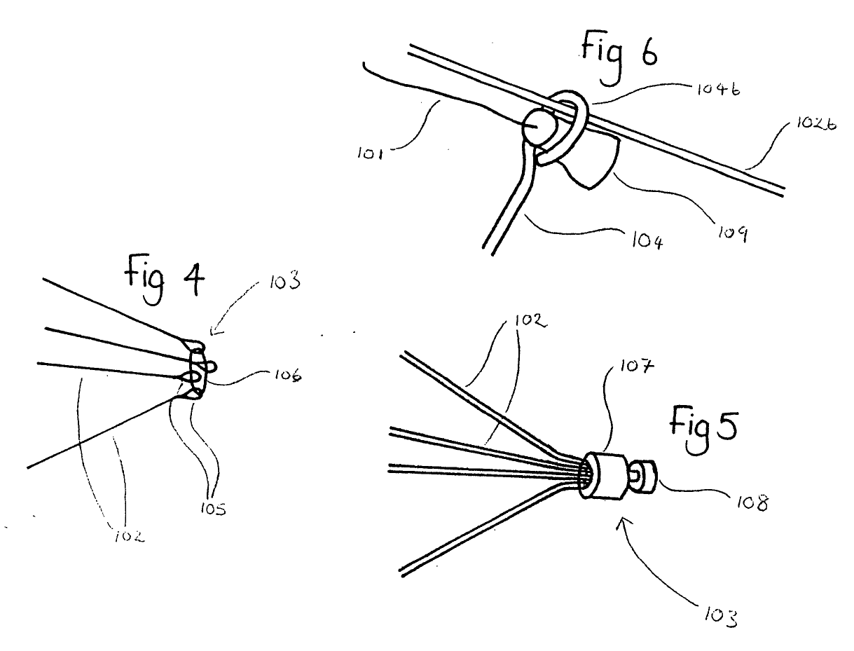

The expansion process of the filter of FIG. 1 may be seen more clearly in FIGS. 3a, 3b and 3c, which show only a first (102a) and second main strut (102b) and a support strut. The first end (104a) of the support strut is attached to the proximal end of the first main strut (102a) and the second opposite end (104b) of the support strut is slidably attached to the second main strut (102b). The proximal end of the first main strut (102a) and first end of the support strut (104a) may be formed as inter-linking wire loops to enable hinging movement of the joint; the second end of the support strut (104b) may be formed as a wire loop encircling the second main strut (102b) to the second end to slide along the length of the second main strut. The suture or thin wire (101) is attached to the second end of the support strut (104b) and may be wound round the length of the second main strut (102b) to avoid tangling of the suture during use. Thus, when this suture (101) is pulled the second end of the support strut (104b) will move proximally and, in doing so, force apart the two main struts (102a, 102b).

In more detail, FIG. 3b shows the point at which the support strut (104) and the second main strut (102b) define a right angle between them; further proximal movement of the second end of the support strut (104b) will cause the two proximal ends of the main struts together rather than away from each other. Further, beyond this point the support strut prevents inwards movement of the two main struts (102a, 102b) and so the structure is resilient to inwards forces applied to the two proximal ends of the main struts. Therefore, when subjected to compressive forces by a vessel wall, the filter structure may become self-supporting and the suture (101) may be removed to allow the filter to remain within the vessel.

FIGS. 4 and 5 display the attachment of the distal end of the main struts (102). FIG. 4 shows a construction where the ends are formed as wire loops (105) which are joined together at a hub (103) by a loop of suture or wire (106). This attachment allows each main strut (102) to move freely about the hub (103). FIG. 5 shows a construction where the main struts (102) are attached to a filter hub (103) formed as an annular member (107) surrounding the distal ends of the main struts (102). During expansion the annular member (107) acts to constrains the distal ends of the main struts (102). Thus, as the main struts (102) are moved apart they deform plastically by bending about the annular member (107). Such plastic deformation ensures that the main struts (102) are intrinsically resilient to inward or outward movement of their proximal ends, so that the filter structure as a whole is self supporting. Such a filter may be expanded to a range of sizes corresponding to the position of the second end of the support strut (not shown), the maximum size being reached when the support strut and the corresponding slidably attached main strut define a right angle between them (as shown in FIG. 36). Thus, the filter may be expanded so that it engages securely with the vessel wall without applying excessive pressure thereupon. The filter hub of FIG. 5 also includes a snare attachment feature (108) which will allow the wire loop of a snare to attach around it to enable retrieval of the filter by its distal end. Such an attachment feature could also be included within the construction of FIG. 4.

FIG. 6 shows an enlarged view of the sliding end of a support strut (104b), the main strut to which it is slidably attached (102b) and the suture (101) used to apply force to the support strut. The distal end of the suture is attached to a deformable plug (109), preferably of frusto-conical shape. This plug (109) serves to apply force to the wire loop at the end of the support strut (104b), and has the particular advantage that if a threshold force is exceeded the plug (109) will deform and pass through the wire loop. During expansion, the sliding end of the support strut (104b) may reach and make contact with the proximal end (not shown) of the main strut to which it is attached (102b). The proximal end will then provide a reaction force against the pulling force; when the pulling force and thus the reaction force exceeds the plug's threshold force, the suture (101) will detach leaving the fully-expanded structure in place. Further, the plug (109) may be used to substantially limit the force that may be applied outwards by the support strut (104) onto the main strut (102b) and thus by the main strut (102b) onto the vessel walls. During expansion the inward reaction force from the wall applied to the main strut will produce a reaction force on the support strut, opposing further expansion. Therefore, this plug (109) may also act as a safety feature to prevent over-stressing the walls of the vena cava as the suture will detach where the pulling force exceeds the threshold value. The plug may be manufactured from a variety of biocompatible polymers and formed for example as a deformable foam.

The ability to expand a filter to a range of radial sizes enables a surgeon to expand a filter so that it engages securely with the vessel walls but does not over-stress them. Thus, the expanded size of the plastically deformable filter may be fine-tuned to the particular size of the vessel in question.

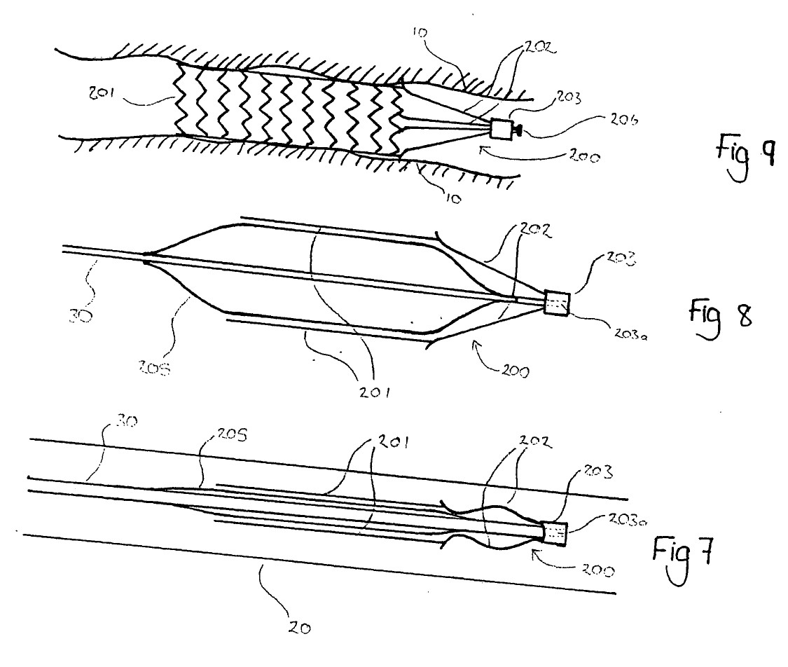

FIGS. 7 to 9 show a further embodiment of the present invention, where the filter (200) may be expanded using a balloon, so that the expansion is controlled remotely from the deployment location. The filter has a plastically deformable stent (201) with a plurality of struts (202) attached to its distal end, the struts themselves being joined at a filter hub (203). The whole construction, including the struts (202), hub (203) and stent (201) are preferably made of the same material. The filter is mounted on a guide wire (30) by a threaded bore (203a) through the centre of the filter hub (203), which cooperates with a screw on the distal end of the guidewire (204). The filter (200) is also mounted on a balloon (205), which extends around and along a distal part of the length of the guide wire. The balloon lies radially interior at least the stent portion of the filter so that expansion of the balloon leads to expansion of the stent, causing the proximal ends of the struts to move radially outwards. FIG. 7 shows the filter (200) in a low profile configuration within the introducer (20) with the struts of the filter (202) and the stent (201) being in a radially compressed configuration. As shown in FIG. 8, the balloon (205) may then be inflated, for example by injecting or introducing a liquid into its lumen as is know in the art. A fluid supply member (not shown) may be mounted on or integral with the guide wire (30) so as to supply fluid to the interior of the balloon; in an exemplary construction a balloon catheter provides both the balloon and the fluid supply member. The balloon (205) preferably comprises an elastic impervious membrane, for example of urethane, allowing the balloon to inflate to a range of sizes. The balloon (205) may also comprise a knitted outer layer designed to reinforce the balloon and limit its maximum radius; such a layer may comprise materials such as Kevlar or spandex. By limiting the maximum radial expansion of the balloon (205) the filter (200) avoids over-stressing the wall of the vena cava. Further, once the filter (200) is expanded to a suitable radius to engage with the vena cava walls (10), the balloon (205) may be deflated so that it returns to a collapsed configuration and may be withdrawn within the introducer for removal, the vena cava filter (200) will then remain within the patient as shown in FIG. 9. As the stent (201) is in substantial contact with the vena cava walls (10) its surface may be drug eluting so that, for example, the filter (200) itself may supply and anti-thrombogenic drugs. The filter shown in FIG. 9 also includes a snare attachment feature (206) provided on its hub (203) to allow retrieval of the filter from its distal end.

In yet a further embodiment, the struts (202) of the filter in FIGS. 7 to 9 may be replaced by a filtering element such comprising a coil, wire mesh, or membrane, with the stent structure (201) used to expand the filtering device (200) and provide support for the filtering element in use.

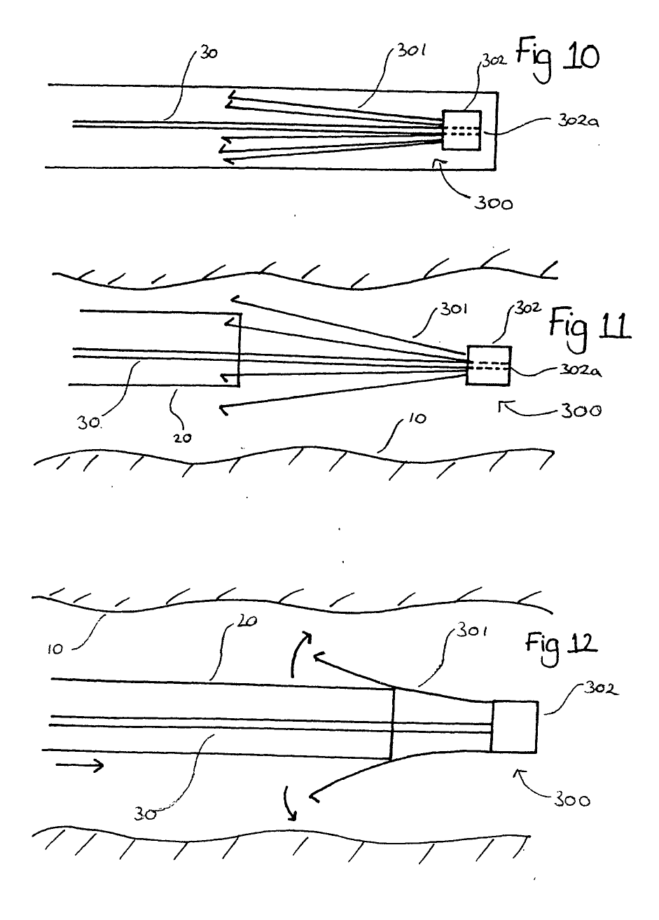

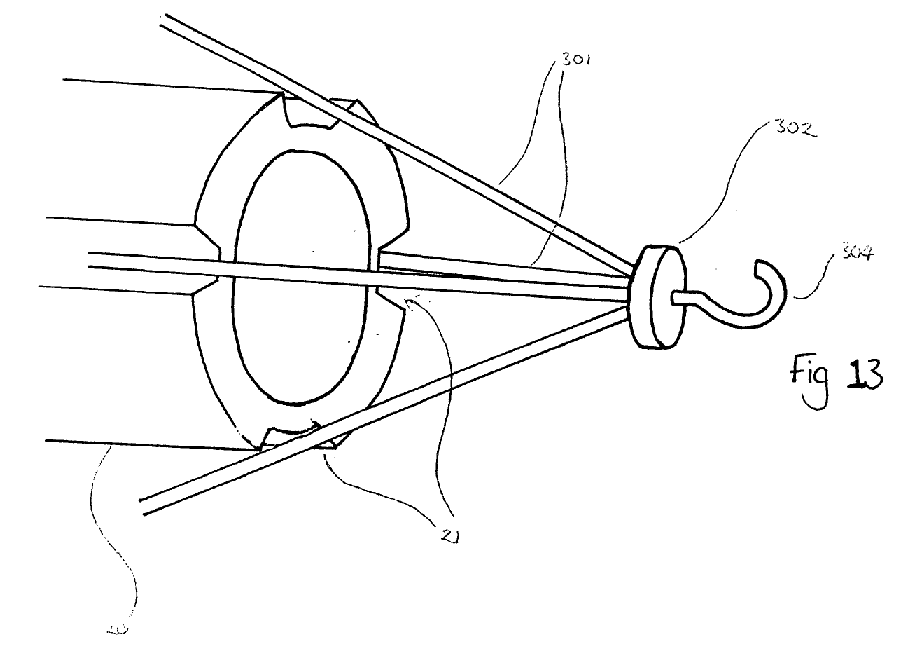

FIGS. 10 to 13 show a still further embodiment of the present invention, where the distal end of the filter comprises a hub (302) to which the distal end of a variety of struts (301) are attached, this hub (302) having a threaded bore (302a) to cooperate with a screw on the distal end of a guidewire (30) for attachment. The hub (302) may be formed as an annular member, restraining the distal ends of the struts within it. FIG. 10 shows the filter (300) compressed into its low profile configuration within the introducer (20); the struts of the filter (301) have a relaxed configuration slightly larger in radial of extent than the introducer (20). By effecting relative movement of the guide wire (30) and introducer (20) the filter (300) exits the distal end of the introducer and assumes its relaxed configuration, as shown in FIG. 11. Preferably the filter struts (301) do not contact the vessel wall (10) in their relaxed configuration. The introducer (20) is then moved distally relative to the filter (300), which is still attached to the guide wire (30). The distal end of the introducer (20) contacts the radial interior of the filter struts (301) forcing them radially outwards and causing them to plastically deform into a progressively expanded configuration. This enables the surgeon to expand the filter (300) by applying opposing forces to the guidewire (30) and introducer (20), thus allowing control over the expansion of the filter (300) remotely of the deployment location. Further, as this method causes the filter (300) to expand to and maintain a desired radius, a suitable force may applied to enable the filter to engage with but not to over-stress the vena cava walls (10).

FIG. 13 displays a still further embodiment of the present invention, which includes a substantially similar filter to that described with reference to FIGS. 10 to 12, and in which strut guiding features (21) extend longitudinally from the distal end of the introducer. These may take the form of longitudinal ridges or channels running away from the distal end of the introducer. As shown in FIG. 13, these features guide the struts (301) outwards, preventing circumferential movement thereof. In particular, this aids in detaching the guide wire (not shown) where the guide wire is attached to the filter hub (302) by means of a screw mechanism. In this fashion, distal force may be applied through the introducer to the filter so that the strut guiding features hold the struts in place while the guide wire is unscrewed from the filter hub (302). This will substantially prevent circumferential movement of the barbs at the proximal ends of the struts, which could cause damage to the vessel walls. Also shown in FIG. 13 is a hook (304) to aid removal of the filter from its distal end using a snare.

It will be understood by those skilled in the art of medical devices that the foregoing embodiments are exemplary and that the teachings may be applied to various filters for implantation within the vasculature. Skilled practitioners will note that further constructions exist within the scope of the present invention to allow a surgeon to control the expansion of the filter from outside the patient. These may include devices whose expansion is mechanically controlled such as those described with reference to FIGS. 1 to 6 or 10 to 13, where a pushing or pulling force is applied by the surgeon over a guidewire or similar control element to cause the filter to adopt an expanded configuration. Further, they may also include fluid controlled devices such as the second embodiment, where the fluid causes expansion by one or more of inflation, cooling and heating of the filter device. Further, they may also include filters that are generally plastically deformable to achieve a desired expanded size, so that the filter may be deformed by the action of the surgeon remote from the filter.

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims. In particular, where specific combinations of features are presented in this specification, which includes the following claims and the drawings, those skilled in the art will appreciate that the features may be incorporated within the invention independently of other disclosed and/or illustrated features.

What is claimed is:

1. Apparatus for the implantation of a filter within the vasculature, comprising: an implantable filter having a structure, said structure being adapted such that under the action of a force applied remotely of the filter it will move after deployment within the vasculature from a radially compressed delivery configuration facilitating navigation through the vasculature, to a radially expanded filtering configuration in which it is in locating engagement with a vessel wall; and means for applying said force remotely of the filter to move said structure from said delivery configuration to said filtering configuration.

2. Apparatus according to claim 1, wherein the structure undergoes plastic deformation in moving from the delivery configuration to the filtering configuration.

3. Apparatus according to claim 1, wherein the structure is adapted such that the radius of the filter in said filtering configuration is controlled by the application of said force.

4. Apparatus according to claim 1, wherein means for applying said force remotely of the filter comprises an inflatable element temporarily located within said structure and means for the injection of fluid thereinto.

5. Apparatus according to claim 1, wherein said means for applying said force remotely of the filter comprises control member moveable in compression.

6. Apparatus according to claim 1, wherein said means for applying said force remotely of the filter comprises control member moveable in tension.

7. Apparatus according to claim 1, wherein the radially compressed delivery configuration of the filter is relaxed and temperature invariant.

8. Apparatus according to claim 1, wherein the structure is adapted to move over-centre between the delivery configuration and the filtering configuration.

9. A method of implantation of a filter within the vasculature, the filter comprising a structure adapted under the action of a force applied remotely of the filter to move after deployment from a radially compressed delivery configuration to a radially expanded filtering configuration in which it is in locating engagement with a vessel wall, the method comprising the steps of deploying the filter in the delivery configuration to or near an implantation site in the vasculature and by intervention of an attendant applying a force to move the structure from the delivery configuration to the filtering configuration.

10. A method according to claim 9, comprising the step of plastically deforming the structure in moving from the delivery configuration to the filtering configuration.

11. A method according to claim 9, comprising the step of controlling the applied force to control the radius of the filter.

12. A method according to claim 9 comprising the steps of introducing at least a portion of an introducer into said patient's vasculature, said introducer comprising a sheath and a control element, said sheath having a lumen and a distal and a proximal end, and harbouring at said distal end said filter in said radially compressed configuration within said lumen; advancing said distal end of said sheath through the vasculature of said patient to a position proximal a deployment location; advancing said filter relative to said sheath so that filter moves beyond the distal end of said sheath and is deployed from the lumen of said sheath; and actuating a portion of said control element exterior to said patient so as to cause said filter to adopt said radially expanded configuration.

13. A method according to claim 12, wherein the structure comprises at least two pivotally interconnected members and at least one slidable member, wherein the control element comprises a flexible pulling member and wherein pulling on the flexible pulling member causes sliding movement of said slidable member effecting relative pivoting of said pivotally interconnected members

14. A method according to claim 12, wherein the step of actuating a portion of said control element comprises the step of moving flexible control member proximally relative to said sheath so as to cause said filter to contact said distal end of said sheath and thereby causing said filter to adopt a radially expanded configuration.

15. An embolic protection filter comprising: a plurality of struts, each having a proximal and a distal end and being attached at their respective distal ends at a filter hub; a support member having a first and a second end, said first end being pivotally attached to a point disposed along the length of a first of said plurality of struts and said second end being slidably attached to a second of said plurality of struts; a flexible pulling member, having a distal and a proximal end, and being attached at its distal end to said support strut at a point spaced away from said first end, so that the application of a force in tension to the pulling member at the proximal end causes the proximal ends of the respective support struts to move apart from one another.

16. A filter according to claim 15, wherein application of a force in tension to the pulling member causes the second end of the support member to slide along the second of the struts to an over centre location along said second of the struts

17. An embolic protection filter according to claim 15, further comprising a plurality of support members, each having a first and a second end, each of said support members being pivotally attached at one end to a point disposed along the length of one of said plurality of struts, being slidably attached at the other end to another of said plurality of struts.

18. Apparatus for the implantation of a filter within the vasculature comprising an embolic protection filter and an introducer, wherein said filter comprises a plurality of struts, each joined at one end in a filter hub, said struts together serving in use to capture emboli; said filter having a radially compressed delivery configuration and a radially expanded filtering configuration and being so adapted that a force external of the filter is required to move the filter from the radially compressed delivery configuration to the radially expanded filtering configuration; wherein said introducer has a distal and a proximal end and comprises: a sheath having a lumen between a proximal and a distal end; and a flexible control member mounted within said sheath, having a distal and a proximal end, wherein said control member is releasably attached at its distal end operable to said filter hub; the filter being harboured within the distal end of the sheath such that relative longitudinal movement in a first sense between the proximal end of the sheath and the proximal end of the control member serves to deploy the filter from the movement and relative longitudinal movement in a second opposite sense between the proximal end of the sheath and the proximal end of the control member serves to move the filter to the radially expanded filtering configuration.

19. Apparatus according to claim 18, wherein said sheath comprises at its distal end at least one engagement feature being shaped to engage with a filter strut on movement of the filter to the radially expanded filtering configuration and to constrain movement of the strut to a radial plane.

20. Method for implanting an embolic protection filter at a deployment location within a patient's vasculature, the filter having an annular vessel engaging portion, having a delivery configuration in which the diameter of said portion is reduced, the method comprising the steps of introducing at least a portion of an introducer into said patient's vasculature, said introducer comprising a sheath and an inflatable member, said sheath having a lumen and a distal and a proximal end, said lumen at said distal end containing said embolic protection filter; advancing said distal end of said sheath through the vasculature of said patient to a position proximal to the deployment location; advancing said filter distally relative to said sheath so that said filter moves beyond the distal end of said sheath and is deployed from the lumen of said sheath and inflating said inflatable member so as to increase the diameter of said vessel engaging portion of filter by an amount controlled by the degree of inflation of the inflatable member to bring said vessel engaging portion of filter into securing engagement with a vessel wall.
